(19) United States
(12) Patent Application Publication
(10) Pub. No.: US 2007/0023052 A1
(43) Pub. Date: Feb. 1, 2007

(54) Oro-genital protection device

(75) Inventor: Paul A. Perry, Las Vegas, NV (US)

Correspondence Address: WEISS & MOY PC, 4204 NORTH BROWN AVENUE, SCOTTSDALE, AZ, 85251, US

(73) Assignee: PAUL A. PERRY & CRAIG E. LAKE

(21) Appl. No.: 11/248,794

(22) Filed: Oct. 13, 2005

Related U.S. Application Data
(63) Continuation-in-part of application No. 11/192,244, filed on Jul. 29, 2005, now abandoned.

(51) Int. Cl. A61F 6/04

(52) U.S. Cl. 128/844; 128/859

(57) ABSTRACT

An oro-genital protection device positionable within the mouth of a wearer. In one embodiment, the device has a gripping area, to facilitate gripping of the device during use by the teeth of a wearer, disposed around a flexible barrier. The flexible barrier, in one embodiment, stretches in response to insertion of a penis, while providing barrier protection against the transmission of STI's. In another embodiment, the device is in the nature of a sheet of flexible material, which may have a flavored area located thereon.

This patent application is a continuation-in-part of a U.S. patent application entitled “Oro-Genital Protection Device” in the name of the same inventor having Ser. No. 11/192,244 and filed Jul. 29, 2005.

### FIELD OF THE INVENTION

The present invention relates generally to devices for protection against sexually transmitted infections and diseases and, more particular, to a device for protection against the transmission of sexually transmitted infections and diseases as a result of oro-genital contact.

### BACKGROUND OF THE INVENTION

There has developed over the years a general awareness of the risk of transmission of sexually transmitted infections and diseases (“STI's”) as a consequence of sexual intercourse. In particular, there is a recognition that unprotected vaginal or anal intercourse can lead to the transmission of STI's between partners. It is further understood that the use of a condom during intercourse can prevent the transmission of STI's.

There is far less awareness of the risk of STI transmission as a consequence of oro-genital contact, including particularly contact between a human mouth and a penis and/or contact between a human mouth and the vaginal area. Such transmission can occur. STI's that are believed to be capable of transmission as a consequence of oro-genital sexual activity include Chlamydia, syphilis, herpes or gonorrhea. On rare occasions, it may be possible to develop genital warts in the mouth. HIV, as well, can be passed through cuts in the mouth or small abrasions.

Lubricated condoms are generally unsuited for such use, because it will lead to the tasting and perhaps ingestion of lubrication that is not intended for such consumption. Un-lubricated condoms may be more suitable, yet these can be difficult to find and, in any event, are of no use where a participant is unwilling to place a condom on his penis. Moreover, condoms are of no assistance with respect to oro-genital contact involving the vagina.

A need therefore exists for an oro-genital protection device that may block the transmission of STI's as a consequence of oro-genital sexual activity. The device should not expose the participant to the risk of ingestion of lubricants. Moreover, the device should not require the positioning of a condom on the penis.

The present invention satisfies these needs and provides other, related, advantages.

### SUMMARY OF THE INVENTION

In accordance with an embodiment of the present invention, an oro-genital protection device is disclosed. The device comprises, in combination: a flexible barrier; a gripping area disposed around the flexible barrier; wherein the gripping area is adapted to be gripped by teeth of a wearer; and wherein the flexible barrier is liquid impermeable.

In accordance with another embodiment of the present invention, an oro-genital protection device is disclosed. The device comprises, in combination: a flexible barrier adapted to be positioned over a genital area; wherein the flexible barrier is liquid impermeable.

### BRIEF DESCRIPTION OF THE DRAWINGS

FIG. 1 is a perspective view of an oro-genital protection device, consistent with an embodiment of the present invention.

FIG. 2 is a side, cross-sectional view of the oro-genital protection device of FIG. 1, in position in the mouth of a wearer, illustrating expansion of the flexible barrier to accommodate the depth of a penis.

FIG. 3A is a perspective view of a protective, flexible barrier, consistent with an embodiment of the present invention.

FIG. 3B is a perspective view of a protective, flexible barrier, consistent with another embodiment of the present invention.

### DETAILED DESCRIPTION OF THE PREFERRED EMBODIMENTS

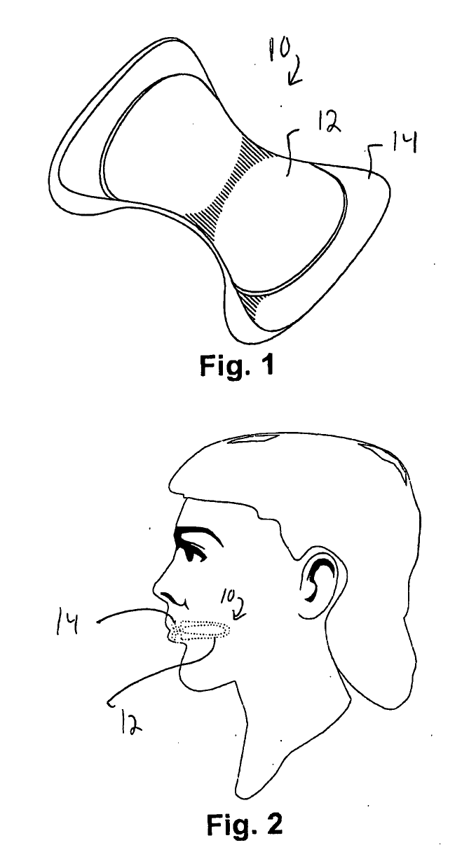

Referring now to FIGS. 1-2, an oro-genital protection device 10 (“device 10) consistent with an embodiment of the present invention is shown. The device 10 comprises a flexible barrier 12 and a gripping area 14, disposed around the flexible barrier 12.

The flexible barrier 12 may be comprised of any material having sufficient qualities of flexibility, to be able to expand to accommodate the entry of a penis into the oral cavity without tearing. The material forming the barrier 12 must also provide sufficient barrier protection to effectively prevent the transmission of STI's therethrough, and thus should be generally liquid impermeable.

Materials considered suitable for the barrier 12 include latex. However, because some individuals may suffer from latex allergies, non-latex materials may also be utilized. These may include vinyl, polyurethane, nitrile, neoprene, or a styrene co-polymer such as tactylite or allegard.

The gripping area 14 should be dimensioned to fit over at least a portion of a wearer's teeth, so that the device 10 may be securely held in position in use. The gripping area 14 should be sufficiently rigid so that the device 10 should not become readily dislodged during sexual activity.

Preferably, the barrier 12 and gripping area 14 are formed as a single piece, to limit the risk of tearing occurring along a seam therebetween. However, it should be noted that it may be desired to provide them as separate components, and to join them through suitable means.

It may be preferred to provide the device 10 in a color that will reduce an awareness of its presence. For example, it may be desired to provide a device 10 (or, at a minimum, the gripping area 14 portion thereof) having a color approximating that of a person's teeth, so that the presence of the device 10 will not be immediately apparent to the wearer's partner. This may be particularly desirable where the non-wearing partner may be hostile to the use of such a device.

In one embodiment, the device 10 may be used by a person involved in oro-genital contact involving a penis. In another embodiment, the device 10 may be used by a person involved in oro-genital contact involving a vagina.

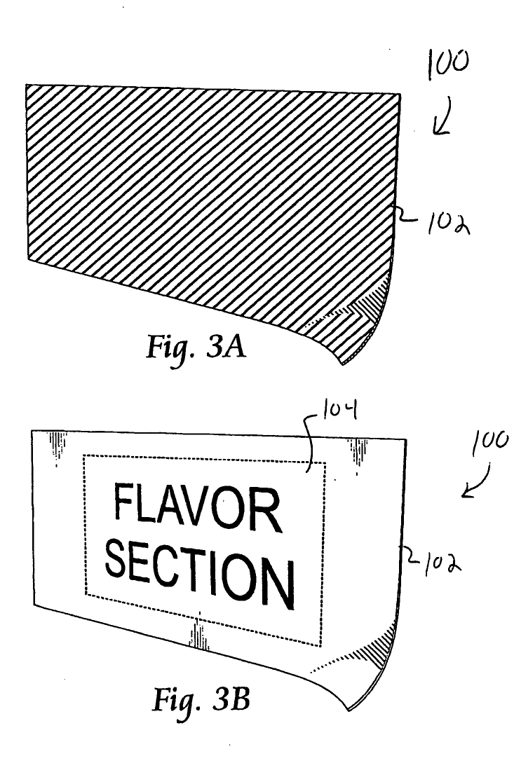

Referring now to FIGS. 3A-3B, an oro-genital protection device 100 (“device 100) consistent with another embodiment of the present invention is shown. The device 100 comprises a flexible barrier 102. Materials considered suitable for the barrier 102 are the same as those reference above with respect to the barrier 12; i.e., latex or non-latex materials such as vinyl, polyurethane, nitrile, neoprene, or a styrene co-polymer such as tactylite or allegard.

In one embodiment, as shown in FIG. 3B, a flavored area 104 is provided on the barrier 102. The flavored area 104 should be coated with a suitable flavoring which may be released upon contact with human saliva. The purpose of the flavored area 104 is to increase the pleasure of the oro-genital activity for the partner who is bringing his or her mouth into contact with the device 100.

In use, the device 100 may be positioned over the genital area. Where the genital area is a penis, the device 100 should be positioned at least so as to cover the external urethral office. Where the genital area is a vagina, the device 100 should be positioned at least so as to cover the vaginal opening, and preferably the vaginal area more generally, including for example the labia.

While the invention has been particularly shown and described with reference to preferred embodiments thereof, it will be understood by those skilled in the art that the foregoing and other changes in form and details may be made therein without departing from the spirit and scope of the invention.

I claim:

1. An oro-genital protection device comprising, in combination:
a flexible barrier;
a gripping area disposed around the flexible barrier;
wherein the gripping area is adapted to be gripped by teeth of a wearer; and
wherein the flexible barrier is liquid impermeable.

2. The device of claim 1 wherein the flexible barrier, when in position in a mouth of a user, permits entry of a penis into the mouth, while sealing an interior surface of the mouth from direct contact therewith.

3. The device of claim 1 wherein the flexible barrier includes latex.

4. The device of claim 1 wherein the flexible barrier comprises a latex-free material.

5. The device of claim 4 wherein the flexible barrier comprises at least one of vinyl, polyurethane, nitrile, neoprene, and a styrene co-polymer.

6. The device of claim 1 wherein the color of at least the gripping area of the device approximately matches that of a typical wearer's teeth.

7. An oro-genital protection device comprising, in combination:
a flexible barrier adapted to be positioned over a genital area;
wherein the flexible barrier is liquid impermeable.

8. The device of claim 7 wherein the flexible barrier includes latex.

9. The device of claim 7 wherein the flexible barrier comprises a latex-free material.

10. The device of claim 9 wherein the flexible barrier comprises at least one of vinyl, polyurethane, nitrile, neoprene, and a styrene co-polymer.

11. The device of claim 7 further comprising a flavored area located on the flexible barrier.
